(19) United States
(12) Patent Application Publication
(10) Pub. No.: US 2011/0282151 A1
(43) Pub. Date: Nov. 17, 2011

(54) IMAGE-BASED LOCALIZATION METHOD AND SYSTEM

(75) Inventors: Karen Irene Trovato, Putnam Valley, NY (US); Aleksandra Popovic, New York, NY (US)

(73) Assignee: KONINKLIJKE PHILIPS ELECTRONICS N.V., EINDHOVEN (NL)

(21) Appl. No.: 13/124,903

(22) PCT Filed: Oct. 12, 2009
(86) PCT No.: PCT/IB2009/054476

§ 371 Date: Jul. 27, 2011

Related U.S. Application Data
(62) Division of application No. 61/106,669, filed on Oct. 20, 2008.

(51) Int. Cl. A61B 1/00

(52) U.S. Cl. 600/117

(57) ABSTRACT

A pre-operative stage of an image-based localization method (30) involves a generation of a scan image (20) illustrating an anatomical region (40) of a body, and a generation of virtual information (21) including a prediction of virtual poses of endoscope (51) relative to an endoscopic path (52) within scan image (20) in accordance with kinematic and optical properties of endoscope (51). An intra-operative stage of the method (30) involves a generation of an endoscopic image (22) illustrating anatomical region (40) in accordance with endoscopic path (52) and a generation of tracking information (23) includes an estimation of poses of endoscope (51) relative to endoscopic path (52) within endoscopic image (22) corresponding to the prediction of virtual poses of endoscope (51) relative to endoscopic path (52) within scan image (20).

The present invention relates to an image-based localization of an anatomical region of a body to provide image-based information about the poses of an endoscope within the anatomical region of a body relative to a scan image of the anatomical region of the body.

Bronchoscopy is an intra-operative procedure typically performed with a standard bronchoscope in which the bronchoscope is placed inside of a patient's bronchial tree to provide visual information of the inner structure.

One known method for spatial localization of the bronchoscope is to use electromagnetic (“EM”) tracking. However, this solution involves additional devices, such as, for example, an external field generator and coils in the bronchoscope. In addition, accuracy may suffer due to field distortion introduced by the metal of the bronchoscope or other object in vicinity of the surgical field. Furthermore, a registration procedure in EM tracking involves setting the relationship between the external coordinate system (e.g., coordinate system of the EM field generator or coordinate system of a dynamic reference base) and the computer tomography (“CT”) image space. Typically, the registration is performed by point-to-point matching, which causes additional latency. Even with registration, patient motion such as breathing can mean errors between the actual and computed location.

Another known method for spatial localization of the bronchoscope is to register the pre-operative three-dimensional (“3D”) dataset with two-dimensional (“2D”) endoscopic images from a bronchoscope. Specifically, images from a video stream are matched with a 3D model of the bronchial tree and related cross sections of camera fly-through to find the relative position of a video frame in the coordinate system of the patient images. The main problem with this 2D/3D registration is complexity, which means it cannot be performed efficiently, in real-time, with sufficient accuracy. To resolve this problem, 2D/3D registration is supported by EM tracking to first obtain a coarse registration that is followed by a fine-tuning of transformation parameters via the 2D/3D registration.

A known method for image guidance of an endoscopic tool involves a tracking of an endoscope probe with an optical localization system. In order to localize the endoscope tip in a CT coordinate system or a magnetic resonance imaging (“MRI”) coordinate system, the endoscope has to be equipped with a tracked rigid body having infrared (“IR”) reflecting spheres. Registration and calibration has to be performed prior to endoscope insertion to be able to track the endoscope position and associate it to the position on the CT or MRI. The goal is to augment endoscopic video data by overlaying a ‘registered’ pre-operative imaging data (CT or MRI).

The present invention is premised on a utilization of a pre-operative plan to generate virtual images of an endoscope within scan image of an anatomical region of a body taken by an external imaging system (e.g., CT, MRI, ultrasound, x-ray and other external imaging systems). For example, as will be further explained herein, a virtual bronchoscopy in accordance with the present invention is a pre-operative endoscopic procedure using the kinematic properties of a bronchoscope or an imaging cannula (i.e., any type of cannula fitted with an imaging device) to generate a kinematically correct endoscopic path within the subject anatomical region, and optical properties of the bronchoscope or the imaging cannula to visually simulate an execution of the pre-operative plan by the bronchoscope or imaging cannula within a 3D model of lungs obtained from a 3D dataset of the lungs.

In the context of the endoscope being a bronchoscope, a path planning technique taught by International Application WO 2007/042986 A2 to Trovato et al. published Apr. 17, 2007, and entitled “3D Tool Path Planning, Simulation and Control System” may be used to generate a kinematically correct path for the bronchoscope within the anatomical region of the body as indicated by the 3D dataset of the lungs.

In the context of the endoscope being an imaging nested cannula, the path planning/nested cannula configuration technique taught by International Application WO 2008/032230 A1 to Trovato et al. published Mar. 20, 2008, and entitled “Active Cannula Configuration For Minimally Invasive Surgery” may be used to generate a, kinematically correct path for the nested cannula within the anatomical region of the body as indicated by the 3D dataset of the lungs.

The present invention is further premised on a utilization of image retrieval techniques to compare the pre-operative virtual image and an endoscopic image of the subject anatomical region taken by an endoscope. Image retrieval as known in the art is a method of retrieving an image with a given property from an image database, such as, for example, the image retrieval technique discussed in Datta, R., Joshi, D., Li, J., and Wang, J. Z. Image retrieval: Ideas, influences, and trends of the newage. ACM Comput. Surv. 40, 2, Article 5 (April 2008). An image can be retrieved from a database based on the similarity with a query image. Similarity measure between images can be established using geometrical metrics measuring geometrical distances between image features (e.g., image edges) or probabilistic measures using likelihood of image features, such as, for example, the similarity measurements discussed in Selim Aksoy, Robert M. Haralick. Probabilistic vs. Geometric Similarity Measures for Image Retrieval, IEEE Conf. Computer Vision and Pattern Recognition, 2000, pp 357-362, vol. 2.

One form of the present invention is an image-based localization method having a pre-operative stage involving a generation of a scan image illustrating an anatomical region of a body, and a generation of virtual information derived from the scan image. The virtual information includes a prediction of virtual poses of the endoscope relative to an endoscopic path within the scan image in accordance with kinematic and optical properties of the endoscope.

In an exemplary embodiment of the pre-operative stage, the scan image and the kinematic properties of the endoscope are used to generate the endoscopic path within the scan image. Thereafter, the optical properties of the endoscope are used to generate virtual video frames illustrating a virtual image of the endoscopic path within the scan image. Additionally, poses of the endoscopic path within the scan image are assigned to the virtual video frames, and one or more image features are extracted from the virtual video frames.

The image-based localization method further has an intra-operative stage involving a generation of an endoscopic image illustrating the anatomical region of the body in accordance with the endoscopic path, and a generation of tracking information derived from the virtual information and the endoscopic image. The tracking information includes an estimation of poses of the endoscope relative to the endoscopic path within the endoscopic image corresponding to the prediction of virtual poses of the endoscope relative to the endoscopic path within the scan image.

In an exemplary embodiment of the intra-operative stage, one or more endoscopic frame features are extracted from each video frame of the endoscopic image. An image matching of the endoscopic frame feature(s) to the virtual frame feature(s) facilitates a correspondence of the assigned poses of the virtual video frames to the endoscopic video frames and therefore the location of the endoscope.

For purposes of the present invention, the term “generating” as used herein is broadly defined to encompass any technique presently or subsequently known in the art for creating, supplying, furnishing, obtaining, producing, forming, developing, evolving, modifying, transforming, altering or otherwise making available information (e.g., data, text, images, voice and video) for computer processing and memory storage/retrieval purposes, particularly image datasets and video frames. Additionally, the phrase “derived from” as used herein is broadly defined to encompass any technique presently or subsequently known in the art for generating a target set of information from a source set of information.

Additionally, the term “pre-operative” as used herein is broadly defined to describe any activity occurring or related to a period or preparations before an endoscopic application (e.g., path planning for an endoscope) and the term “intra-operative” as used herein is broadly defined to describe as any activity occurring, carried out, or encountered in the course of an endoscopic application (e.g., operating the endoscope in accordance with the planned path). Examples of an endoscopic application include, but are not limited to, a bronchoscopy, a colonscopy, a laparascopy, and a brain endoscopy.

In most cases, the pre-operative activities and intra-operative activities will occur during distinctly separate time periods. Nonetheless, the present invention encompasses cases involving an overlap to any degree of pre-operative and intra-operative time periods.

Furthermore, the term “endoscope” is broadly defined herein as any device having the ability to image from inside a body. Examples of an endoscope for purposes of the present invention include, but are not limited to, any type of scope, flexible or rigid (e.g., arthroscope, bronchoscope, choledochoscope, colonoscope, cystoscope, duodenoscope, gastroscope, hysteroscope, laparoscope, laryngoscope, neuroscope, otoscope, push enteroscope, rhinolaryngoscope, sigmoidoscope, sinuscope, thorascope, etc.) and any device similar to a scope that is equipped with an image system (e.g., a nested cannula with imaging). The imaging is local, and surface images may be obtained optically with fiber optics, lenses, or miniaturized (e.g. CCD based) imaging systems.

The foregoing form and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

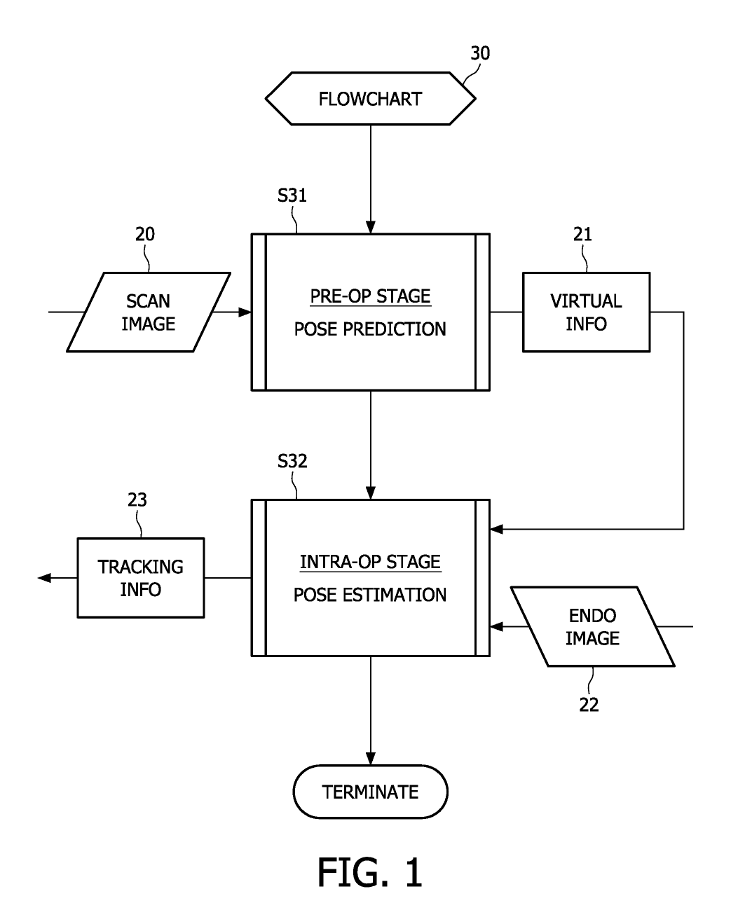

FIG. 1 illustrates a flowchart representative of one embodiment of an image-based localization method of the present invention.

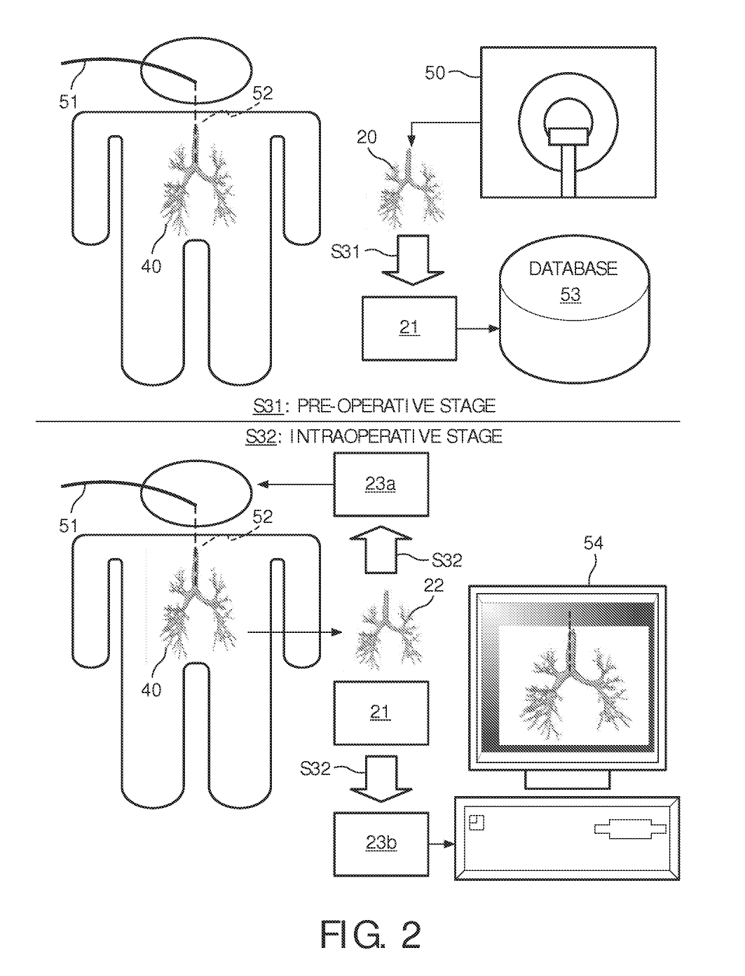

FIG. 2 illustrates an exemplary bronchoscopy application of the flowchart illustrated in FIG. 1.

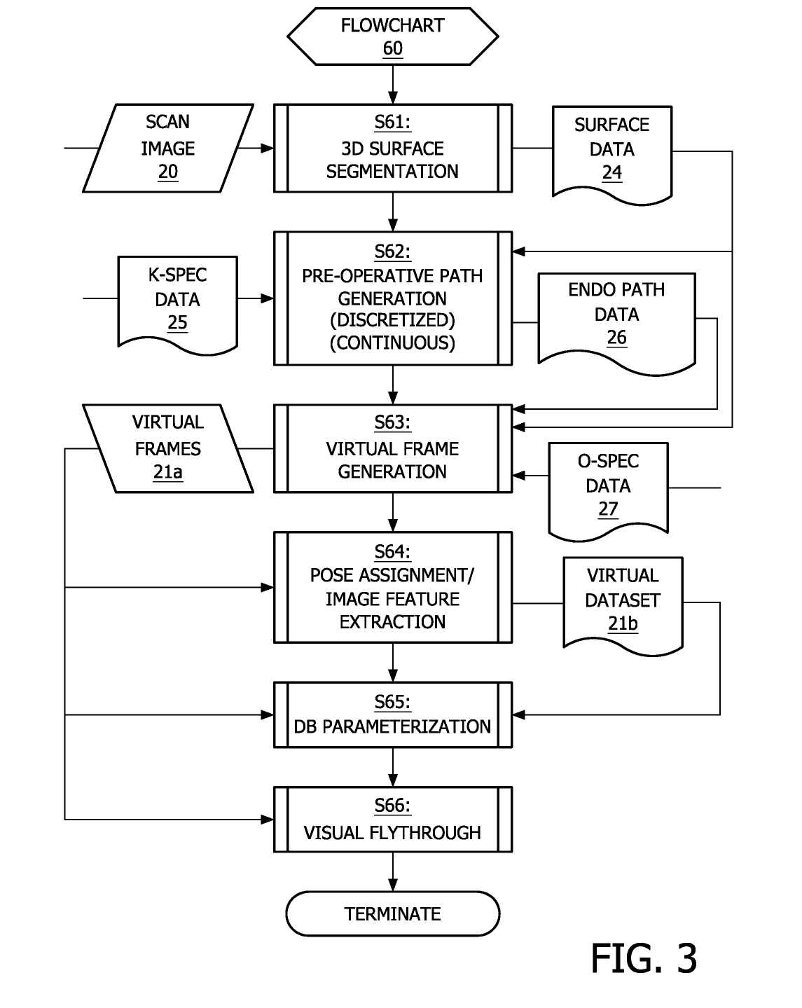

FIG. 3 illustrates a flowchart representative of one embodiment of a pose prediction method of the present invention.

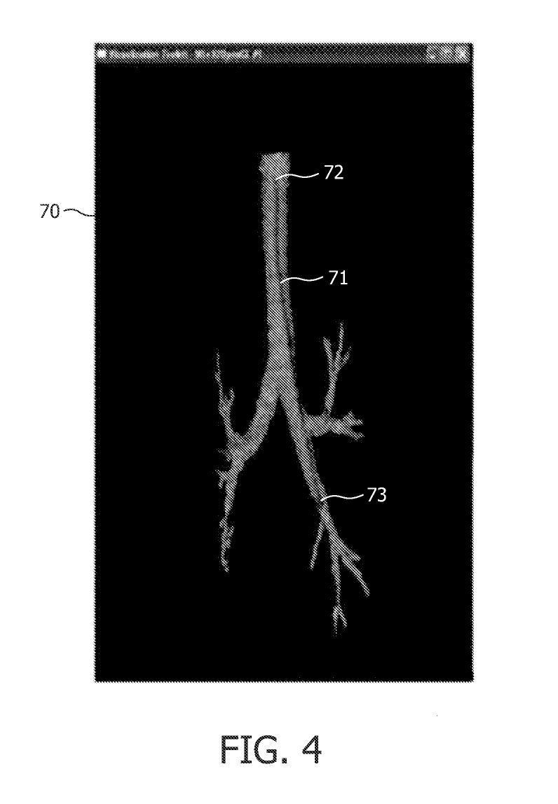

FIG. 4 illustrates an exemplary endoscopic path generation for a bronchoscope in accordance with the flowchart illustrated in FIG. 3.

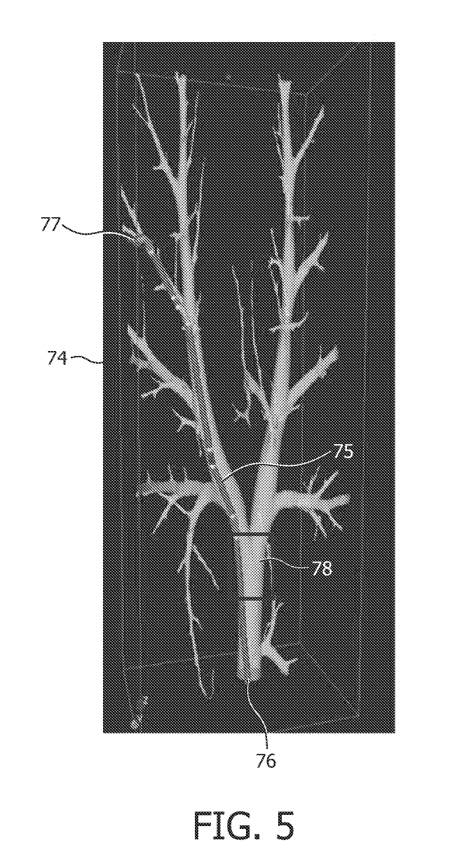

FIG. 5 illustrates an exemplary endoscopic path generation for a nested cannula in accordance with the flowchart illustrated in FIG. 3.

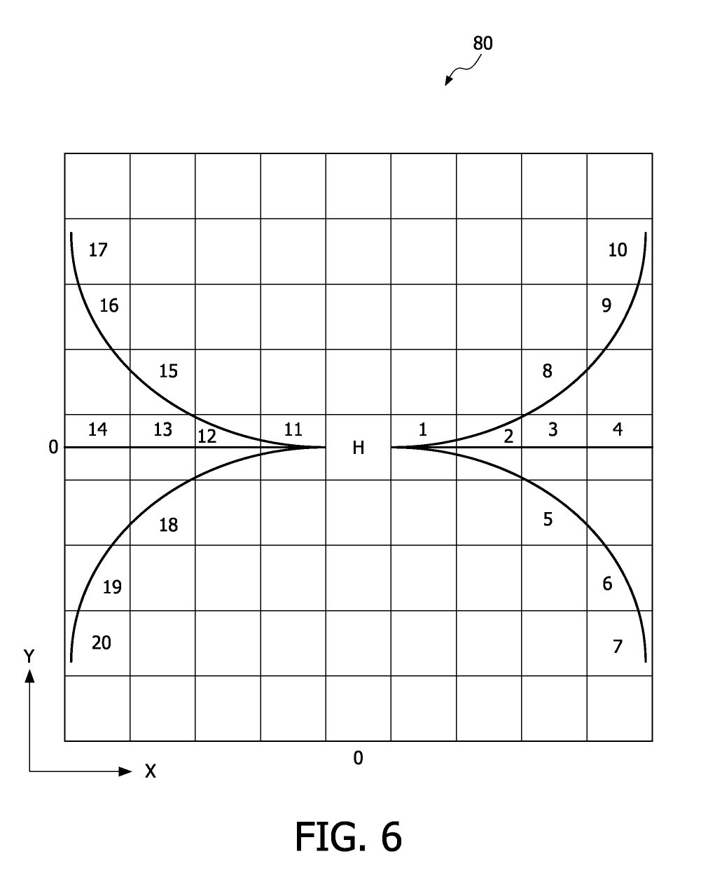

FIG. 6 illustrates an exemplary coordinate space and 2-D projection of a non-holonomic neighborhood in accordance with the flowchart illustrated in FIG. 3.

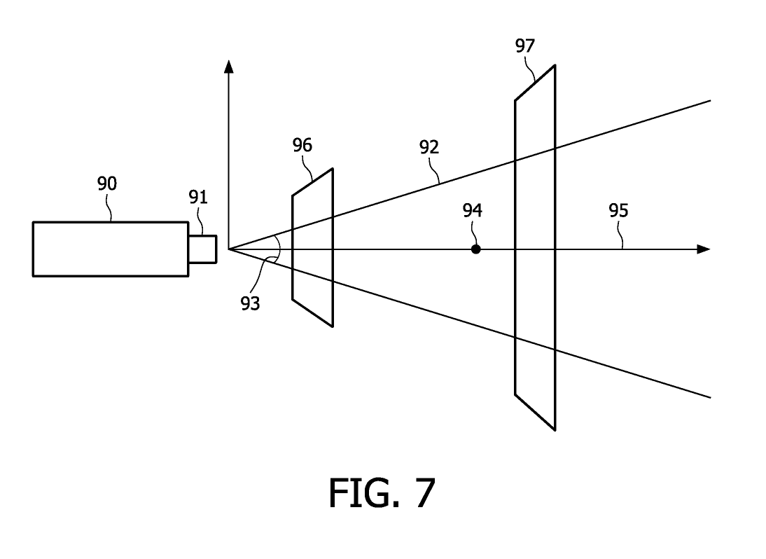

FIG. 7 illustrates an exemplary optical specification data in accordance with the flowchart illustrated in FIG. 3.

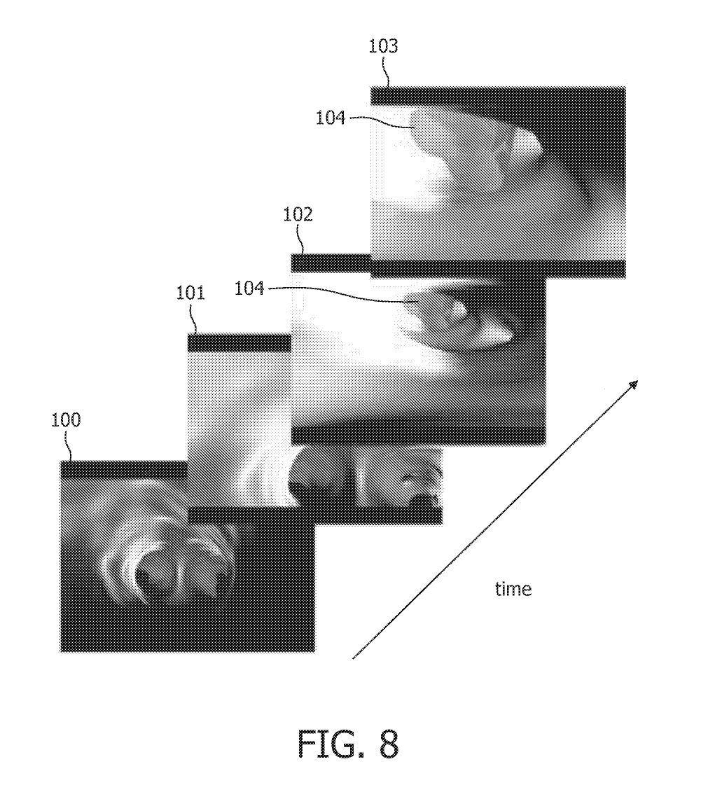

FIG. 8 illustrates an exemplary virtual video frame generation in accordance with the flowchart illustrated in FIG. 3.

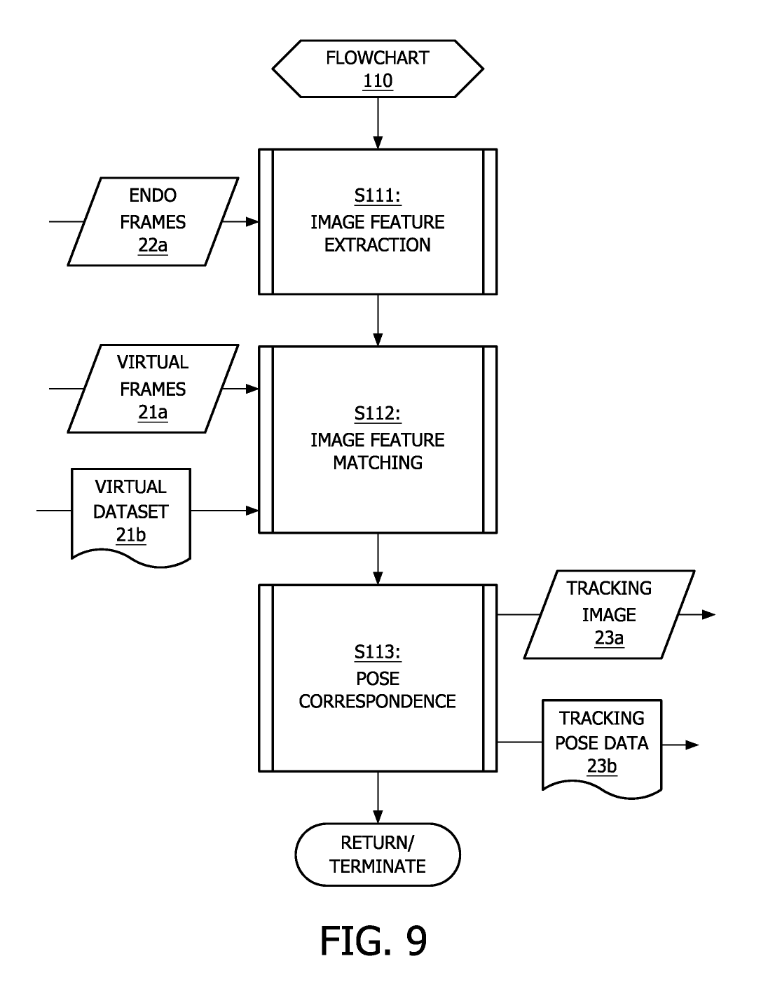

FIG. 9 illustrates a flowchart representative of one embodiment of a pose estimation method of the present invention.

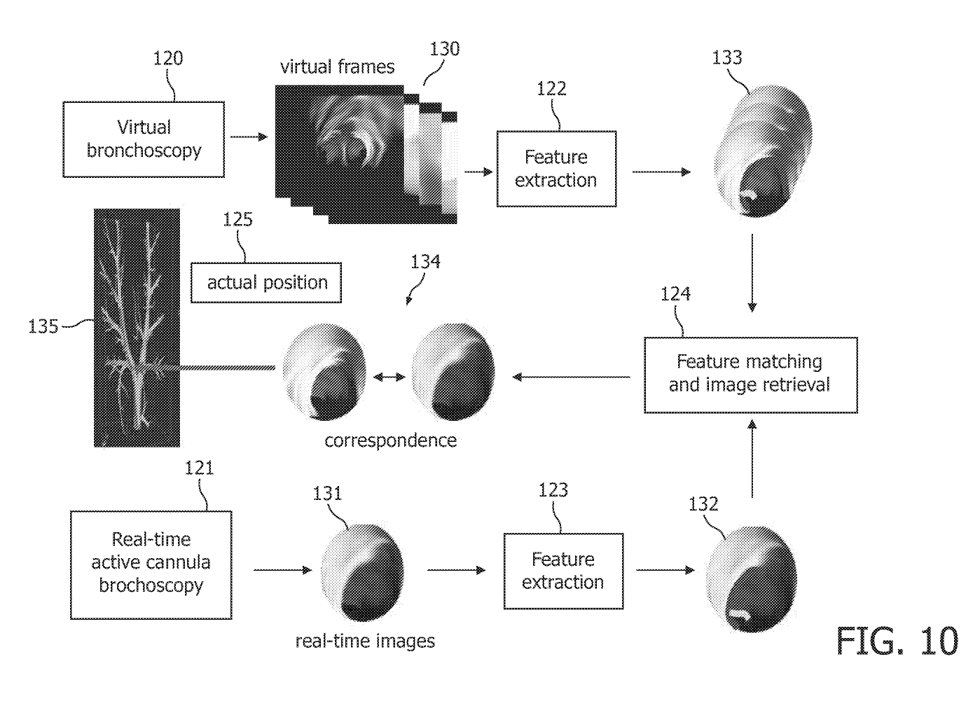

FIG. 10 illustrates an exemplary tracking of an endoscope in accordance with the flowchart illustrated in FIG. 9.

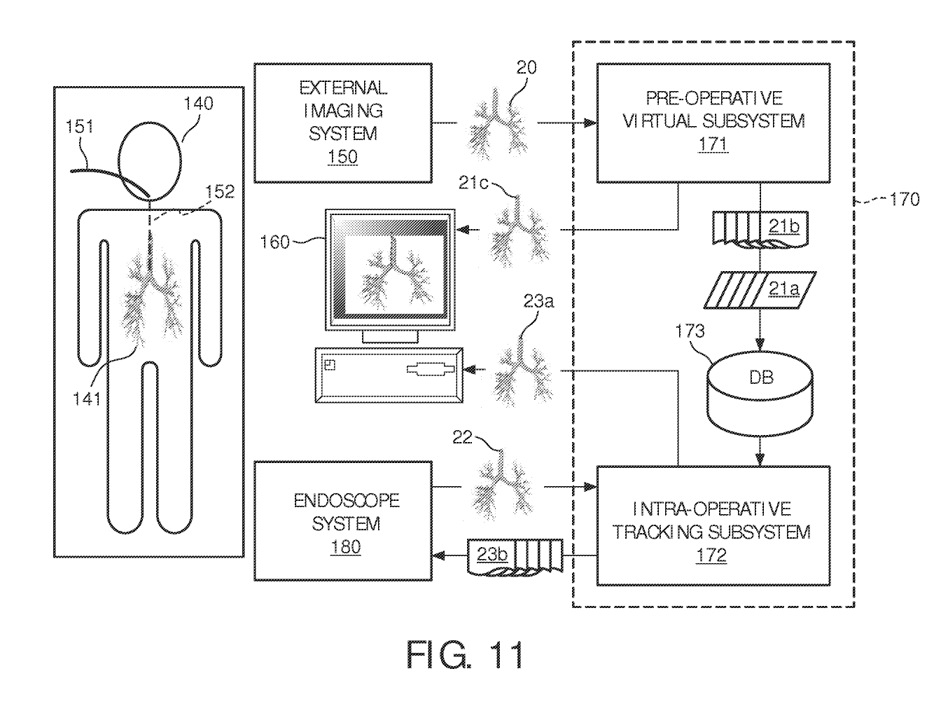

FIG. 11 illustrates one embodiment of an image-based localization system of the present invention.

A flowchart 30 representative of an image-based localization method of the present invention is shown in FIG. 1. Referring to FIG. 1, flowchart 30 is divided into a pre-operative stage S31 and an intra-operative stage S32.

Pre-operative stage S31 encompasses an external imaging system (e.g., CT, MRI, ultrasound, x-ray, etc.) scanning an anatomical region of a body, human or animal, to obtain a scan image 20 of the subject anatomical region. Based on a possible need for diagnosis or therapy during intra-operative stage S32, a simulated optical viewing by an endoscope of the subject anatomical region is executed in accordance with a pre-operative endoscopic procedure. Virtual information detailing poses of the endoscope predicted from the simulated viewing is generated for purposes of estimating poses of the endoscope within an endoscopic image of the anatomical region during intra-operative stage S32 as will be subsequently described herein.

For example, as shown in the exemplary pre-operative stage S31 of FIG. 2, a CT scanner 50 may be used to scan bronchial tree 40 of a patient resulting in a 3D image 20 of bronchial tree 40. A virtual bronchoscopy may be executed thereafter based on a need to perform a bronchoscopy during intra-operative stage S32. Specifically, a planned path technique using scan image 20 and kinematic properties of an endoscope 51 may be executed to generate an endoscopic path 52 for endoscope 51 through bronchial tree 40, and an image processing technique using scan image 20 and optical properties of endoscope 51 may be executed to simulate an optical viewing by endoscope 51 of bronchial tree 40 relative to the 3D space of scan image 20 as the endoscope 51 virtually traverses endoscopic path 52. Virtual information 21 detailing predicted virtual locations (x,y,z) and orientations (α,θ,φ) of endoscope 51 within scan image 20 derived from the optical simulation may thereafter be immediately processed and/or stored in a database 53 for purposes of the bronchoscopy.

Referring again to FIG. 1, intra-operative stage S32 encompasses the endoscope generating an endoscopic image 22 of the subject anatomical region in accordance with an endoscopic procedure. To estimate the poses of the endoscope within the subject anatomical region, virtual information 21 is referenced to correspond the predicted virtual poses of the endoscope within scan image 20 to endoscopic image 22. Tracking information 23 detailing the results of the correspondence is generated for purposes of controlling the endoscope to facilitate compliance with the endoscopic procedure and/or of displaying of the estimated poses of the endoscope within endoscopic image 22.

For example, as shown in the exemplary intra-operative stage S32 of FIG. 2, endoscope 51 generates an endoscopic image 22 of bronchial tree 40 as endoscope 51 is operated to traverse endoscopic path 52. To estimate locations (x,y,z) and orientations (α,θ,φ) of endoscope 51 in action, virtual information 21 is referenced to correspond the predicted virtual poses of endoscope 51 within scan image 20 of bronchial tree 40 to endoscopic image 22 of bronchial tree 40. Tracking information 23 in the form of a tracking pose data 23a is generated for purposes for providing control data to an endoscope control mechanism (not shown) of endoscope 51 to facilitate compliance with the endoscopic path 52. Additionally, tracking information 23 in the form of tracking pose image 23a is generated for purposes of displaying the estimated poses of endoscope 51 within bronchial tree 40 on a display 54.

The preceding description of FIGS. 1 and 2 teach the general inventive principles of the image-based localization method of the present invention. In practice, the present invention does not impose any restrictions or any limitations to the manner or mode by which flowchart 30 is implemented. Nonetheless, the following descriptions of FIGS. 3-10 teach an exemplary embodiment of flowchart 30 to facilitate a further understanding of the image-based localization method of the present invention.

A flowchart 60 representative of a pose prediction method of the present invention is shown in FIG. 3. Flowchart 60 is an exemplary embodiment of the pre-operative stage S31 of FIG. 1.

Referring to FIG. 3, a stage S61 of flowchart 60 encompasses an execution of a 3D surface segmentation of an anatomical region of a body as illustrated in scan image 20, and a generation of 3D surface data 24 representing the 3D surface segmentation. Techniques for a 3D surface segmentation of the subject anatomical region are known by those having ordinary skill in the art. For example, a volume of a bronchial tree can be segmented from a CT scan of the bronchial tree by using a known marching cube surface extraction to obtain an inner surface image of the bronchial tree needed for stages S62 and S63 of flowchart 60 as will be subsequently explained herein.

Stage S62 of flowchart 60 encompasses an execution of a planned path technique (e.g., a fast marching or A* searching technique) using 3D surface data 24 and specification data 25 representing kinematic properties of the endoscope to generate a kinematically customized path for the endoscope within scan image 20. For example, in the context of endoscope being a bronchoscope, a known path planning technique taught by International Application WO 2007/042986 A2 to Trovato et al. dated Apr. 17, 2007, and entitled “3D Tool Path Planning, Simulation and Control System”, an entirety of which is incorporated herein by reference, may be used to generate a kinematically customized path within scan image 20 as represented by the 3D surface data 24 (e.g., a CT scan dataset). FIG. 4 illustrates an exemplary endoscopic path 71 for a bronchoscope within a scan image 70 of a bronchial tree. Endoscopic path 71 extends between an entry location 72 and a target location 73.

Also by example, in the context of the endoscope being an imaging nested cannula, the path planning/nested cannula configuration technique taught by International Application WO 2008/032230 A1 to Trovato et al. published Mar. 20, 2008, and entitled “Active Cannula Configuration For Minimally Invasive Surgery”, an entirety of which is incorporated herein by reference, may be used to generate a kinematically customized path for the imaging cannula within the subject anatomical region as represented by the 3D surface data 24 (e.g., a CT scan dataset). FIG. 5 illustrates an exemplary endoscopic path 75 for an imaging nested cannula within an image 74 of a bronchial tree. Endoscopic path 75 extends between an entry location 76 and a target location 77.

Continuing in FIG. 3, endoscopic path data 26 representative of the kinematically customized path is generated for purposes of stage S63 as will be subsequently explained herein and for purposes of conducting the intra-operative procedure via the endoscope during intra-operative stage 32 (FIG. 1). A pre-operative path generation method of stage S62 involves a discretized configuration space as known in the art, and endoscopic path data 26 is generated as a function of the coordinates of the configuration space traversed by the applicable neighborhood. For example, FIG. 6 illustrates a three-dimensional non-holonomic neighborhood 80 of seven (7) threads 81-87. This encapsulates the relative position and orientation that can be reached from the home position H at the orientation represented by thread 81.

The pre-operative path generation method of stage S62 preferably involves a continuous use of a discretized configuration space in accordance with the present invention, so that the endoscopic path data 26 is generated as a function of the precise position values of the neighborhood across the discretized configuration space.

The pre-operative path generation method of stage S62 is preferably employed as the path generator because it provides for an accurate kinematically customized path in an inexact discretized configuration space. Further the method enables a 6 dimensional specification of the path to be computed and stored within a 3D space. For example, the configuration space can be based on the 3D obstacle space such as the anisotropic (non-cube voxels) image typically generated by CT. Even though the voxels are discrete and non-cubic, the planner can generate continuous smooth paths, such as a series of connected arcs. This means that far less memory is required and the path can be computed quickly. Choice of discretization will affect the obstacle region, and thus the resulting feasible paths, however. The result is a smooth, kinematically feasible path, in a continuous coordinate system for the endoscope. This is described in more detail in U.S. Patent Application Ser. Nos. 61/075,886 and 61/099,233 to Trovato et al. filed, respectively, Jun. 26, 2008 and Sep. 23, 2008, and entitled “Method and System for Fast Precise Planning”, an entirety of which is incorporated herein by reference.

Referring back to FIG. 3, a stage S63 of flowchart 60 encompasses a sequential generation of 2D cross-sectional virtual video frames 21a illustrating a virtual image of the endoscopic path within scan image 20 as represented by 3D surface data and endoscopic path data 26 in accordance with the optical properties of the endoscope as represented by optical specification data 27. Specifically, a virtual endoscope is advanced on the endoscopic path and virtual video frames 21a are sequentially generated at pre-determined path points of the endoscopic path as a simulation of video frames of the subject anatomical region that would be taken by a real endoscope advancing the endoscopic path. This simulation is accomplished in view of the optical properties of the physical endoscope.

For example, FIG. 7 illustrates several optical properties of an endoscope 90 relevant to the present invention. Specifically, the size of a lens 91 of endoscope 90 establishes a viewing angle 93 of a viewing area 92 having a focal point 94 along a projection direction 95. A front clipping plane 96 and a back clipping plane 97 are orthogonal to projection direction 95 to define the visualization area of endoscope 90, which is analogous to the optical depth of field. Additional parameters include the position, angle, intensity and color of the light source (not shown) of endoscope 90 relative to lens 91. Optical specification data 27 (FIG. 3) may indicate one or more the optical properties 91-97 for the applicable endoscope as well as any other relevant characteristics.

Referring back to FIG. 3, the optical properties of the real endoscope are applied to the virtual endoscope. At any given path point in the simulation, knowing where the virtual endoscope is looking within scan image 20, what area of scan image 20 is being focused on by the virtual endoscope, the intensity and color of light emitted by the virtual endoscope and any other pertinent optical properties facilitates a generation of a virtual video frame as a simulation of a video frame taken by a real endoscope at that path point.

For example, FIG. 8 illustrates four (4) exemplary sequential virtual video frames 100-103 taken from an area 78 of path 75 shown in FIG. 5. Each frame 100-103 was taken at pre-determined path point in the simulation. Individually, virtual video frames 100-103 illustrate a particular 2D cross-section of area 78 simulating an optical viewing of such 2D cross-section of area 78 taken by an endoscope within the subject bronchial tree.

Referring back to FIG. 3, a stage S64 of flowchart 60 encompasses a pose assignment of each virtual video frame 21a. Specifically, the coordinate space of scan image 20 is used to determine a unique position (x,y,z) and orientation (α,θ,φ) of each virtual video frame 21a within scan image 20 in view of the position and orientation of each path point utilized in the generation of virtual video frames 21a.

Stage S64 further encompasses an extraction of one or more image features from each virtual video frame 21a. Examples of the feature extraction includes, but is not limited to, an edge of a bifurcation and its relative position to the view field, an edge shape of a bifurcation, an intensity pattern and spatial distribution of pixel intensity (if optically realistic virtual video frames were generated). The edges may be detected using simple known edge operators (e.g., Canny or Laplacian), or using more advanced known algorithms (e.g., a wavelet analysis). The bifurcation shape may be analyzed using known shape descriptors and/or shape modeling with principal component analysis. By further example, as shown in FIG. 8, these techniques may be used to extract the edges of frames 100-103 and a growth 104 shown in frames 102 and 103.

The result of stage S64 is a virtual dataset 21b representing, for each virtual video frame 21a, a unique position (x,y,z) and orientation (α,θ,φ) in the coordinate space of the pre-operative image 20 and extracted image features for feature matching purposes as will be further explained subsequently herein.

A stage S65 of flowchart 60 encompasses a storage of virtual video frames 21a and virtual pose dataset 21b within a database having the appropriate parameter fields.

A stage S66 of flowchart 60 encompasses a utilization of virtual video frames 21a to executes of visual fly-through of an endoscope within the subject anatomical region for diagnosis purposes.

Referring again to FIG. 3, a completion of flowchart 60 results in a parameterized storage of virtual video frames 21a and virtual dataset 21b whereby the database will be used to find matches between virtual video frames 21a and video frames of endoscopic image 22 (FIG. 1) of the subject anatomical region generated and to correspond the unique position (x,y,z) and orientation (α,θ,φ) of each virtual video frame 21a to a matched endoscopic video frame.

Further to this point, FIG. 9 illustrates a flowchart 110 representative of a pose estimation method of the present invention. During the intra-operative procedure, a stage S111 of flowchart 110 encompasses an extraction of image features from each 2D cross-sectional video frame 22a of endoscopic image 22 (FIG. 1) obtained from the endoscope of the subject anatomical region. Again, examples of the feature extraction includes, but is not limited to, an edge of a bifurcation and its relative position to the view field, an edge shape of a bifurcation, an intensity pattern and spatial distribution of pixel intensity (if optically realistic virtual video frames were generated). The edges may be detected using simple known edge operators (e.g., Canny or Laplacian), or using more advanced known algorithms (e.g., a wavelet analysis). The bifurcation shape may be analyzed using known shape descriptors and/or shape modeling with principal component analysis.

Stage S112 of flowchart 110 further encompasses an image matching of the image features extracted from virtual video frames 21a to the image features extracted from endoscopic video frames 22a. A known searching technique for finding two images with the most similar features using defined metrics (e.g., shape difference, edge distance etc) can be used to match the image features. Furthermore, to gain time efficiency, the searching technique may be refined to use real-time information about previous matches of images in order to constrain the database search to a specific area of the anatomical region. For example, the database search may be constrained to points and orientations plus or minus 10 mm from the last match, preferably first searching along the expected path, and then later within a limited distance and angle from the expected path. Clearly, if there is no match, meaning a match within acceptable criteria, then the location data is not valid, and the system should register an error signal.

A stage S113 of flowchart 110 further encompasses a correspondence of the position (x,y,z) and orientation (α,θ,φ) of a virtual video frame 21a to an endoscopic video frame 22a matching the image feature(s) of the virtual video frame 21a to thereby estimate the poses of the endoscope within endoscopic image 22. More particularly, feature matching achieved in stage 5112 enables a coordinate correspondence of the position (x,y,z) and orientation (α,θ,φ) of each virtual video frame 21a within a coordinate system of the scan image 20 (FIG. 1) of subject anatomical region to one of the endoscopic video frames 22a as an estimation of the poses of the endoscope within endoscopic image 22 of the subject anatomical region.

This pose correspondence facilitates a generation of a tracking pose image 23b illustrating the estimated poses of the endoscope relative to the endoscopic path within the subject anatomical region. Specifically, tracking pose image 23a is a version of scan image 20 (FIG. 1) having an endoscope and endoscopic path overlay derived from the assigned poses of the endoscopic video frames 22a.

The pose correspondence further facilitates a generation of tracking pose data 23a representing the estimated poses of the endoscope within the subject anatomical region Specifically, the tracking pose data 23b can have any form (e.g., command form or signal form) to used in a control mechanism of the endoscope to ensure compliance to the planned endoscopic path.

For example, FIG. 10 illustrates virtual video frames 130 provided by a virtual bronchoscopy 120 performed by use of an imaging nested cannula and an endoscopic video frame 131 provided by an intra-operative bronchoscopy performed by use of the same or kinematically and optically equivalent imaging nested cannula. Virtual video frames 130 are retrieved from an associated database whereby previous or real-time extraction 122 of image features 133 (e.g., edge features) from virtual video frames 130 and an extraction 123 of an image feature 132 from an endoscopic video frame 131 facilitates a feature matching 124 of a pair of frames. As a result, a coordinate space correspondence 134 enables a control feedback and a display of an estimated position and orientation of an endoscope 125 within bronchial tubes illustrated in the tracking pose image 135.

As prior positions and orientations of the endoscope are known and each endoscopic video frame 131 is being made available in real-time, the ‘current location’ should be nearby, therefore narrowing the set of candidate images 130. For example, there may be many similar looking bronchi. ‘Snapshots’ along each will create a large set of plausible, but possibly very different locations. Further, for each location even a discretized subset of orientations will generate a multitude of potential views. However, if the assumed path is already known, the set can be reduced to those likely x,y,z locations and likely α,θ,φ (rx,ry,rz) orientations, with perhaps some variation around the expected states. In addition, based on the prior ‘matched locations’, the set of images 130 that are candidates is restricted to those reachable within the elapsed time from those prior locations. The kinematics of the imaging cannula restrict the possible choices further. Once a match is made between a virtual frame 130 and the ‘live image’ 131, the position and orientation tag from the virtual frame 130 gives the coordinates in pre-operative space of the actual orientation of the imaging cannula in the patient.

FIG. 11 illustrates an exemplary system 170 for implementing the various methods of the present invention. Referring to FIG. 11, during a pre-operative stage, an imaging system external to a patient 140 is used to scan an anatomical region of patent 140 (e.g., a CT scan of bronchial tubes 141) to provide scan image 20 illustrative of the anatomical region. A pre-operative virtual subsystem 171 of system 170 implements pre-operative stage S31 (FIG. 1), or more particularly, flowchart 60 (FIG. 3) to display a visual flythrough 21c of the relevant pre-operative endoscopic procedure via a display 160, and to store virtual video frames 21a and virtual dataset 21b into a parameterized database 173. The virtual information 21a/b details a virtual image of an endoscope relative to an endoscopic path within the anatomical region (e.g., a endoscopic path 152 of a simulated bronchoscopy using an image nested cannula 151 through bronchial tree 141).

During an intra-operative state, an endoscope control mechanism (not shown) of system 180 is operated to control an insertion of the endoscope within the anatomical region in accordance with the planned endoscopic path therein. System 180 provides endoscopic image 22 of the anatomical region to an intra-operative tracking subsystem 172 of system 170, which implements intra-operative stage S32 (FIG. 1), or more particularly, flowchart 110 (FIG. 9) to display tracking image 23a to display 160, and/or to provide tracking pose data 23b to system 180 for control feedback purposes. Tracking image 22a and tracking pose data 23b are collectively informative of an endoscopic path of the physical endoscope through the anatomical region (e.g., a real-time tracking of a imaging nested cannula 151 through bronchial tree 141). In the case where system 172 fails to achieve a feature match between virtual video frames 21a and endoscopic video frames (not shown), tracking pose data 23a will contain an error message signifying the failure.

While various embodiments of the present invention have been illustrated and described, it will be understood by those skilled in the art that the methods and the system as described herein are illustrative, and various changes and modifications may be made and equivalents may be substituted for elements thereof without departing from the true scope of the present invention. In addition, many modifications may be made to adapt the teachings of the present invention to entity path planning without departing from its central scope. Therefore, it is intended that the present invention not be limited to the particular embodiments disclosed as the best mode contemplated for carrying out the present invention, but that the present invention include all embodiments falling within the scope of the appended claims.

1. An image-based localization method (30), comprising:
generating a scan image (20) illustrating an anatomical region (40) of a body;
generating an endoscopic path (52) within the scan image (20) in accordance with kinematic properties of an endoscope (51); and
generating virtual video frames (21a) illustrating a virtual image of the endoscopic path (52) within the scan image (20) in accordance with optical properties of the endoscope (51).

2. The image-based localization method (30) of claim 1, further comprising:
assigning poses of the endoscopic path (52) within the scan image (20) to the virtual video frames (21a); and
extracting at least one virtual frame feature from each virtual video frame (21a).

3. The image-based localization method (30) of claim 2, further comprising:
generating a parameterized database (54) including the virtual video frames (21a) and a virtual pose dataset (21b) representative of the pose assignments of the endoscope (51) and the extracted at least one virtual frame feature.

4. The image-based localization method (30) of claim 1, further comprising:
executing a visual fly-through of the virtual video frames (21a) illustrating predicted poses of the endoscope (51) relative to the endoscopic path (52) within the anatomical region (40).

5. The image-based localization method (30) of claim 2, further comprising:
generating an endoscopic image (22) illustrating the anatomical region (40) of the body in accordance with the endoscopic path (52); and
extracting at least one endoscopic frame feature from each endoscopic video frame (22a) of the endoscopic image (22).

6. The image-based localization method (30) of claim 5, further comprising:
image matching the at least one endoscopic frame feature to the at least one virtual frame feature; and
corresponding assigned poses of the virtual video frames (21a) to the endoscopic video frames (22a) in accordance with the image matching.

7. The image-based localization method (30) of claim 6, further comprising:
generating a tracking pose image (23a) illustrating estimated poses of the endoscope (51) within the endoscopic image (22) in accordance with the pose assignments of the endoscopic video frames (22a); and
providing the tracking pose images frames (23a) to a display (56).

8. The image-based localization method (30) of claim 6, further comprising:
generating a tracking pose data (23b) representing the pose assignments of the endoscopic video frames (22a); and
providing the tracking pose data (23b) to an endoscope control mechanism (180) of the endoscope (51).

9. The image-based localization method (30) of claim 1, wherein the endoscopic path (52) is generated as a function of precise position values of neighborhood nodes within a discretized configuration space (80) associated with the scan image (20).

10. The image-based localization method (30) of claim 1, wherein the endoscope (51) is selected from a group including a bronchoscope and an imaging cannula.

11. An image-based localization method (30), comprising:
generating a scan image (20) illustrating an anatomical region (40) of a body; and
generating virtual information (21) derived from the scan image (20),
wherein the virtual information (21) includes a prediction of virtual poses of an endoscope (51) relative to an endoscopic path (53) within the scan image (20) in accordance with kinematic and optical properties of the endoscope (51).

12. The image-based localization method (30) of claim 11, further comprising:
generating an endoscopic image (22) illustrating the anatomical region (40) of the body in accordance with the endoscopic path (52); and
generating tracking information (23) derived from the virtual information and the endoscopic image (22),
wherein the tracking information (23) includes an estimation of poses of the endoscope (51) relative to the endoscopic path (52) within the endoscopic image (22) corresponding to the prediction of virtual poses of the endoscope (51) relative to the endoscopic path (52) within the scan image (20).

13. A image-based localization system, comprising;
a pre-operative virtual subsystem (171) operable to generate virtual information (21) derived from a scan image (20) illustrating an anatomical region (40) of the body,
wherein the virtual information (21) includes a prediction of virtual poses of an endoscope (51) relative to an endoscopic path (53) within the scan image (20) in accordance with kinematic and optical properties of the endoscope (51); and
an intra-operative tracking subsystem (172) operable to generate tracking information (23) derived from the virtual information (21) and an endoscopic image (22) illustrating the anatomical region (40) of the body in accordance with the endoscopic path (52),
wherein the tracking information (23) includes an estimation of poses of the endoscope (51) relative to the endoscopic path (52) within the endoscopic image (22) corresponding to the prediction of virtual poses of the endoscope (51) relative to the endoscopic path (52) within the scan image (20).

14. The image-based localization system of claim 13, further comprising:
a display (160),
wherein the intra-operative tracking subsystem (172) is further operable to provide a tracking pose image (23a) illustrating the estimated poses of the endoscope (51) relative to the endoscopic path (52) within the endoscopic image (22) to the display (56).

15. The image-based localization system of claim 13, further comprising:
an endoscope control mechanism (180),
wherein the intra-operative tracking subsystem (172) is further operable to provide a tracking pose data (23b) representing the estimated poses of the endoscope (51) relative to the endoscopic path (52) within the endoscopic image (22) to the endoscopic control mechanism (180).
